(19) United States
(12) Patent Application Publication
(10) Pub. No.: US 2008/0045796 A1
(43) Pub. Date: Feb. 21, 2008

(54) HISTOFLUORESCENT STAIN COMPOSITION FOR ENDOSCOPY

(75) Inventors: Akira YAMAMOTO, Tokyo (JP); Yusuke IIMORI, Tokyo (JP); Mizue SAZE, Tokyo (JP); Mariko ISHIGURO, Tokyo (JP); Pilryon LEE, Tokyo (JP); Hiroyuki SASAKI, Kanagawa (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/758,688

(22) Filed: Jun. 6, 2007

(30) Foreign Application Priority Data
Jun. 6, 2006 (JP) 2006-156925

(51) Int. Cl. A61B 1/00; A61K 49/00

(52) U.S. Cl. 600/160; 424/9.6

(57) ABSTRACT

A fluorescent stain which has a function of clarifying the shape of the surface of the gastrointestinal lumen in the visible light wavelength region, and simultaneously, emitting fluorescence by being excited by light of a specific wavelength, and which is also biologically safe and is suitable for endoscopic observation, is provided. A histofluorescent stain composition for endoscopy, containing a compound represented by the following Formula (1): 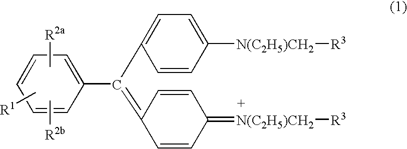
wherein R¹ represents a hydrogen atom or a hydroxyl group; R³ represents a phenyl group or methyl group, to which R^{2c} is bound; one to three of R^{2a}, R^{2b} and R^{2c} represent —SO₃M (wherein M represents an alkali metal atom or an alkaline earth metal atom), or —SO₃NH₄, and when one represents —SO₃⁻, the others represent hydrogen atoms is provided.

### BACKGROUND OF THE INVENTION

1. Field of the Invention

The present invention relates to a histofluorescent stain composition used in diagnoses by means of an endoscope.

2. Description of the Related Art

Diagnostic techniques using an endoscope are widely applied in the diagnosis of diseases such as, in particular, cancers, gastrointestinal ulcers, ulcerative colitis and the like, mainly on the basis of the gastrointestinal endoscopic examination for the upper gastrointestinal tract and the lower gastrointestinal tract. The detection of anomalies (lesions) in the tissues by such endoscopic examination generally involves observation with a visible light endoscope at a magnification of about 10 to 500 times without using a staining agent. Meanwhile, there is a method referred to as chromoendoscopy, in which endoscopic observation is made in the state where a solution containing a dye has been disseminated over tissue surfaces. Since it is possible to clearly observe the shape of the surface of the gastrointestinal lumen by this chromoendoscopy, even microscopic lesions can be easily discovered by the change of color. Examples of the endoscope that can be used for chromoendoscopy include a visible light endoscope and a fluorescent endoscope.

Examples of the dye mainly used in chromoendoscopy include indigo carmine (Masahiro Tada, et al., “Clinical Gastroenterology, Vol. 7, No. 2, 1992”), which is used for observation by the contrast method, for the staining of the gastrointestinal lumen under visible light; acriflavin and fluorescein (Gastroenterology 2004, Vol. 127, No. 3, p. 706-713) for fluorescent staining; and the like.

In the diagnosis of cancer or the like, it is important to observe the surface of biological tissues as well as the inside of biological tissues. With regard to the method of observing the inside of biological tissues, generally a method of slicing a micro part of a tissue collected by biopsy or the like in the laboratory, and staining and observing the micro part is practiced. For a method of observing the inside of biological tissues in situ, for example, MRI, PET, CT, soft X-ray and the like are applied for the observation of the whole body. With regard to the gastrointestinal endoscopy, endoscopes involving autofluorescent reaction of biological tissues have been commercialized. When a light having a specific wavelength is irradiated to a biological tissue, there occurs autofluorescence of endogenous fluorescent substances in the tissue, and thus, visual observation of normal areas and lesion areas can be made possible by means of the intensity differences and the spectra.

In order to make a diagnosis of, for example, gastrointestinal cancer using a conventional endoscope, it has been required to determine the lesion area in an empirical manner by observation, incise a tissue specimen therefrom, and make a diagnosis by means of tissue staining in a separate laboratory. However, it is now possible to observe the inside of a tissue without incising the tissue, by using a recently developed confocal endoscope.

In general, a confocal imaging system is a technique of obtaining clear images of the inside of a tissue without mechanical cutting, by placing a pinhole in front of the detector and detecting the light reflected only from the focal plane inside the tissue. Typically, the confocal image system involves scanning a laser light across a tissue stained with a fluorescent substance, and observing a fluorescent image thereof. The confocal imaging system generally needs fluorescent staining agents.

Since the confocal endoscope employing the confocal imaging system has both a normal observation optical system and a confocal observation optical system, it is useful from the viewpoints that screening of the lesion area is possible, and that the observation of cells in situ through optical tissue slicing without cutting out the cells becomes possible, which is less invasive.

Confocal endoscopes that are currently being marketed employ blue laser light having a wavelength of 488 nm as a light source for dye excitation. It is an important property required for a fluorescent dye to be used in the confocal endoscopes for medical purposes that the fluorescent dye has no toxicity or mutagenecity against the body. Therefore, for the present, the fluorescent dye that can be used for confocal endoscopes for medical purposes is limited to fluorescein for intravenous injection for fundus angiography, and acriflavin that is used as an antibiotic substance (Gastrointestinal Endoscopy Clin of N Am., 2005, Vol. 12, p. 715-731).

With regard to the light source used for the confocal endoscopes, diversification of the wavelength is anticipated in the future. For example, since a light in the region from red to infrared has higher permeability to biological tissues than blue light, it is believed to give confocal images at positions deeper than the surface of the biological tissues. There is available IndoCyanine Green (ICG) as an infrared fluorescent compound for diagnosis which has been approved for administration to human body, and it is mainly used for the examination of hepatic function and fundus angiography. If IndoCyanine Green is used as a staining agent for confocal endoscopy, it is not possible to achieve clear contrast compared to, for example, the fluorescein described in Gastroenterology 2004, Vol. 127, No. 3, p. 706-713. Furthermore, if IndoCyanine Green is to be used as a fluorescent reagent for endoscopy, it has a problem of being highly toxic compared to the fluorescein.

### SUMMARY OF THE INVENTION

Accordingly, it is an object of the present invention to provide a histofluorescent stain composition for endoscopy, which composition 1) has low biotoxicity, 2) confers contrast to emphasize the irregularities on the tissue surface even under a white light source, 3) is excited by a light source in the red wavelength region and emits fluorescence, 4) penetrates into the tissue and confers an observation image of the internal structure of the tissue, which is recognizable as a fluorescence image by a confocal endoscope, and 5) has different chromophilic areas from fluorescein, and can clearly give contrast images of blood vessels, lamina propria mucosae, collagen fibers and the like in the gastrointestinal lumen.

The inventors of the present invention have investigated in various aspects while focusing on the safety of a stain, stainability with respect to visible light, generation of fluorescence by a light source in the red wavelength region, stainability in fluorescence observation and the like, and as a result, found that a compound of the following Formula (1), which may be represented by Fast Green FCF, Brilliant Blue FCF, or the like, satisfies the requirements described above. In addition, they also discovered that such compound is useful as a fluorescent stain for staining the inside of a tissue in confocal endoscopy, and that the stained images obtained therefrom are characteristic and useful for the detection of lesions in early stages.

Thus, the present invention is to provide a histofluorescent stain composition for endoscopy, comprising a compound represented by the following formula (1): 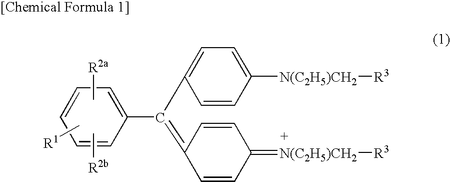
wherein R¹ represents a hydrogen atom or a hydroxyl group; R³ represents a phenyl group or a methyl group, to which R^{2c} is bound; one to three of R^{2a}, R^{2b} and R^{2c} represent —SO₃M (wherein M represents an alkali metal atom or an alkaline earth metal atom), or —SO₃NH₄, provided that when one represents —SO₃⁻, the others represent hydrogen atom.

It is another object of the present invention to provide a method of diagnosing with an endoscope by administrating the composition comprising the compound represented by the Formula (1), and observing the tissue fluorescent stained by the composition with an endoscope.

It is another object of the present invention to provide use of the compound represented by the Formula (1) for the manufacture of a histofluorescent stain for endoscopy.

(1) For the observation of the gastrointestinal tract under a conventional endoscope using a white light source, the histofluorescent stain of the present invention, which is a deep-blue dye, results in strong contrast compared to red dyes such as fluorescein and the like.

(2) For the observation with an endoscope which combines a white light endoscope and a confocal endoscope, the stain of the present invention confers deep-blue contrast with respect to white light, while exhibiting fluorescence with respect to red exciting light. Thus, the discovery of lesion areas and the obtainment of confocal images thereof can be provided by a single dye upon using the endoscope described above.

(3) The compound of Formula (1) exhibits fluorescence, and has a sulfonic group in the molecule, thus staining well the intercellular spaces or connective tissues upon staining of the intestine or the like. The stainability of the dye is different from fluorescein, and thus, when used in combination with such a dye, more information concerning tissues can be obtained in the observation of confocal images.

(4) It is important that the histofluorescent stain of the present invention allows direct observation of the condition of the connective tissues, particularly with respect to tumors having developed fibrous tissues. The vascular walls of the arteries and veins are also chromophilic, so that the blood vessels inside tumors can be observed.

(5) Since the histofluorescent stain of the present invention gives fluorescence having a wavelength near red, upon observing the fluorescence from a focal plane in deep parts, the decay of the fluorescence due to the tissue through which the fluorescence passes is less than that of fluorescein, and therefore clear confocal images of the tissues in the deep parts below the surface can be obtained.

(6) The histofluorescent stain of the present invention exhibits only faint fluorescence for any wavelength when dissolved in water. However, when disseminated into the gastrointestinal lumen, strong fluorescence is emitted. Thus, the stain has an advantage that upon staining by dissemination, washing is not needed, and observation of low background fluorescence is possible.

(7) Accordingly, when the histofluorescent stain of the present invention is used, visualization of the surface of lesion areas and the inside of tissues can be achieved simultaneously under observation by visible light, fluorescence and confocal endoscopies, without collecting the tissues, and the stained images are sharp and clear, thus the stain being useful in the diagnosis of gastrointestinal diseases and the like.

### BRIEF DESCRIPTION OF THE DRAWINGS

FIG. 1 is a diagram showing the excitation and fluorescence spectrum of Fast Green FCF;

FIG. 2 is a diagram showing the excitation and fluorescence spectrum of IndoCyanine Green;

FIG. 3 is a picture showing the results of confocal microscopic observation of mouse large intestine which has been fluorescent stained with Fast Green FCF;

FIG. 4 is a picture showing the results of confocal microscopic observation of mouse large intestine which has been fluorescent stained with Fluorescite;

FIG. 5 is a picture showing the results of confocal microscopic observation of mouse large intestine which has been double stained with Fast Green FCF and Fluorescite;

FIG. 6 is a picture showing the results of confocal microscopic observation of the lumen of mouse large intestine which has been fluorescent stained with Fast Green FCF;

FIG. 7 is a picture showing the results of confocal microscopic observation of the lumen of mouse large intestine which has been fluorescent stained with IndoCyanine Green;

FIG. 8 is a picture showing the stainability of the inside of a tissue with Fast Green FCF;

FIG. 9 is a picture showing the results of HE staining of a tissue specimen;

FIG. 10 is a picture showing the results of staining of an area 40 μm deep from the luminal surface of duodenum using Fast Green FCF;

FIG. 11 is a picture showing the results of staining of an area about 10 μm deep from the luminal surface of large intestine using Fast Green FCF;

FIG. 12 is a picture showing the results of staining of an area 15 μm deep from the surface layer of liver using Fast Green FCF;

FIG. 13 is a picture showing the results of staining of an area 100 μm deep from the luminal surface of esophagus using Brilliant Blue FCF;

FIG. 14 is a set of pictures serially taken at every 5.0 μm from an area 10 μm deep from the surface layer of a rat tumor which has been stained with Fast Green FCF;

FIG. 15 is a set of pictures serially taken at every 5.0 μm from an area 10 μm deep from the surface layer of a rat tumor which has been stained with Monascus Yellow;

FIG. 16 is a picture showing the results of confocal microscopic observation of mouse large intestine stained with Patent Blue;

FIG. 17 is a picture showing the HE-stained images of mouse large intestine;

FIG. 18 is a picture showing the results of confocal microscopic observation of mouse large intestine stained with Guinea Green; and

FIG. 19 is a picture showing the HE-stained images of mouse large intestine.

### DESCRIPTION OF THE PREFERRED EMBODIMENTS

The present disclosure relates to subject matter contained in Japanese Patent Application No. 2006-156925, filed on Jun. 6, 2006, which is expressly incorporated herein by reference in its entirety.

Examples of the endoscope of the present invention include medical endoscopies such as a gastrointestinal endoscope, a respiratory endoscope, a vascular endoscope, a celioscope, a thoracoscope, and the like. Among these, the gastrointestinal endoscope is particularly preferred. According to the present invention, visible light endoscopes include conventional endoscopes including all of the endoscopes observing under visible light, regular endoscopes, magnifying endoscopes, and chromoendoscopes observing visible light. Meanwhile, fluorescent endoscopes include endoscopes measuring the fluorescence generated by irradiation of an excitation light, and also include magnifying fluorescent endoscopes. Furthermore, confocal endoscopes refer to endoscopes equipped with a confocal imaging system. In addition, a confocal endoscope conventionally has both a normal observation optical system and a confocal observation system.

The histofluorescent stain of the present invention contains a compound represented by the Formula (1) described above. In the Formula (1), R¹ represents a hydrogen atom or a hydroxyl group. When R¹ represents a hydroxyl group, the bonding position is preferably meta or about para with respect to the methine group of Formula (1). R³ is a phenyl group or methyl group to which R^{2c} is bound. Among R^{2a}, R^{2b} and R^{2c}, one to three of them represent —SO₃M (wherein M represents an alkali metal atom or an alkaline earth metal atom), or —SO₃NH₄, but it is preferable that one or two of them are —SO₃M or —SO₃NH₄. One of R^{2a}, R^{2b} and R^{2c} is —SO₃⁻. Furthermore, the remaining R^{2a}, R^{2b} and R^{2c} are hydrogen atoms. Here, the alkali metal atom represented by M may be exemplified by sodium, potassium, or lithium, but sodium is particularly preferred. The alkaline earth metal atom may be exemplified by calcium, magnesium or the like, but calcium is particularly preferred.

Specific preferable examples of such compound represented by the Formula (1) include Fast Green FCF, Brilliant Blue FCF, Guinea Green B, Patent Blue VF, Patent Blue NA, Light Green SF, Alphazurine FG, Patent Blue CA (calcium salt type of Patent Blue NA), and the like.

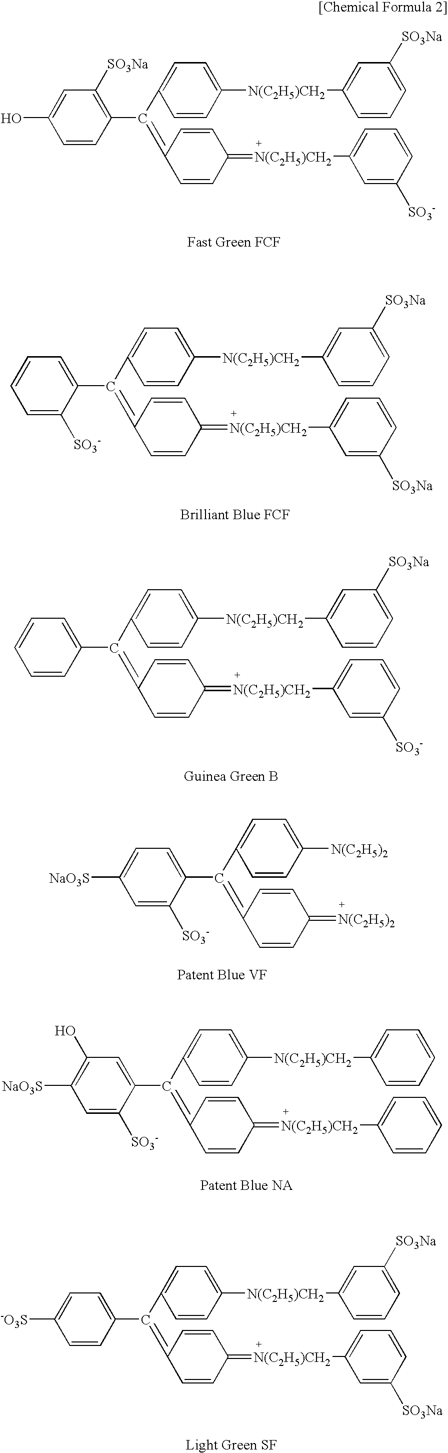

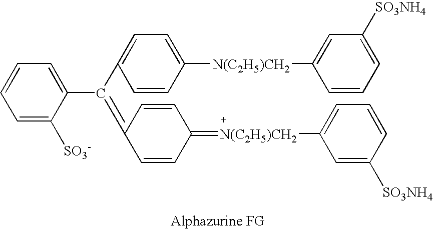

Fast Green FCF is known as Green No. 3; Brilliant Blue FCF as Blue No. 1; Guinea Green B as Green No. 402; Patent Blue NA as Blue No. 202; Light Green SF as Green No. 205; Alphazurine FG as Blue No. 205; and Patent Blue CA as Blue No. 203. These are widely used as colorants for cosmetic or pharmaceutical products. Thus, the safety of these components has been established. However, it is not known as to whether these compounds emit fluorescence, and it is not known at all that when applied to tissues, these compounds exhibit clear fluorescent images of the inside of the tissues.

Commercially available products of the Fast Green FCF include, for example, Fast Green FCF of Wako Pure Chemical Industries, Ltd. Commercially available products of the Brilliant Blue FCF include, for example, Food Blue No.1 of Kiriya Chemical Co., Ltd., Food Blue No. 1 of San-Ei Gen F.F.I., Inc., and the like. Commercially available products of the Guinea Green B include Guinea Green B manufactured by Sigma-Aldrich Corp. Commercially available products of the Patent Blue VF include Patent Blue VF manufactured by Sigma-Aldrich Corp. Commercially available products of the Light Green SF include Light Green SF manufactured by Wako Pure Chemical Industries, Ltd. Commercially available products of Alphazurine FG include Erioglaucine manufactured by Sigma-Aldrich Corp.

The content of the compound of Formula (1) in the tissue stain of the present invention is preferably 0.01 to 70% by weight, more preferably 0.01 to 50% by weight, and particularly preferably 0.01 to 20% by weight, from the viewpoints of stainability and the clarity of stained images.

The tissue stain of the present invention can be administered by any means selected from oral administration, direct administration into the gastrointestinal tract, and submucosal administration. The tissue stain may be in any form, such as liquid, granule, tablet or the like. In the case of disseminating into the gastrointestinal tract or administering submucosally, liquid is preferred, while in the case of oral administration, liquid, granule, tablet and the like are preferred.

The tissue stain of the present invention may have various components blended in according to the form (formulation). For example, a viscosity agent, a thickening agent, a surfactant, a sweetening agent, a preservative, a flavoring agent, a pH adjusting agent, water and the like may be blended in.

The pH adjusting agent is an agent adjusting the pH to 5 to 9, and examples thereof include hydrochloric acid, phosphoric acid, citric acid, malic acid, acetic acid and salts thereof, sodium hydroxide, potassium hydroxide, sodium hydrogen carbonate, tetrasodium pyrrolate and the like.

Furthermore, ethanol, water and the like may be added as a solvent. In the case of tablets, components known to be used for tablets, such as a binding agent, a disintegrating agent and the like, can be used.

Since the tissue stain of the present invention can stain tissues to be blue to greenish in color, the stain is useful as a tissue stain for conventional white light endoscopic observation. The endoscope used herein is a conventional endoscope or a magnifying endoscope, and is useful for endoscopic observation at a magnifying power of 10 to 500 times.

Fast Green FCF can be excited at, for example, 628 nm and have the fluorescence detected at 652 nm. Brilliant Blue FCF can be excited at, for example, 629 nm and have the fluorescence detected at 650 nm. Guinea Green B can be excited at, for example, 624 nm and have the fluorescence detected at 635 nm. Patent Blue VF can be excited at, for example, 641 nm and have the fluorescence detected at 654 nm. Light Green SF can be excited at, for example, 633 nm and have the fluorescence detected at 652 nm. Alphazurine FG can be excited at, for example, 633 nm and have the fluorescence detected at 643 nm.

The stainability for tissues differs between normal tissues, tissues in a pre-cancerous state or tissue in which a tumor is present. In the case of staining normal tissues, the cellular membrane and intercellular space of the epithelial cells in the small intestine, large intestine or the like are stained. On the other hand, in the observation of malignant tumors, features of individual cells including (1) nuclear predominance due to an increase in the nuclear volume, (2) increased amount of chromatin in the nucleus and hyperchromatism, (3) increases in the volume and number of nucleoli, (4) karyological anomaly in the number or form of chromosomes, (5) basophilic staining of cells, and its involvement in the proliferative properties of the cells, and the like are observed. Furthermore, in tumor cells, a large number of nuclear chromatins assemble close to the nuclear membrane, thus endoplasmic reticulum being simplified. Moreover, mitochondria become irregular and inhomogenous in size, and there occur an increased amount of filamentous structures in the cells. When the tissue stain composition of the present invention is used, first, collagen fibers and elastic fibers, muscle layers, and the like are favorably stained. Thus, the stain composition is very useful from the aspect that any information for the areas having a potential to develop tumors can be quickly known. Next, since the stain composition tends to emit fluorescence only for bound tissues, only the information that is needed to know can be obtained, thus making the significance of the stain composition high.

As indicated by the following Examples, the compound of Formula (1) is characterized not only by its emission of fluorescence, but also by clarity of the fluorescent stained images of the inside of tissues, as compared to other food dyes. Therefore, the stain of the present invention is useful as a histofluorescent stain for endoscopy.

In addition, if the endoscope employing a confocal optical system has both a normal observation optical system and a confocal observation optical system, it is possible to diagnose the surface as well as the inside of tissues without extracting the lesion area tissues by visually observing the lesion area by observation under normal light, then upon reaching the lesion of question, observing fluorescent stained cross-sectional images of the inside of the tissue (for example, up to 250 μm below the surface) with a confocal endoscope. That is, the shape of cells or nuclei of a living organism can be observed in a viable state. As a result, diagnosis of gastrointestinal diseases such as pre-cancerous state, cancer, ulcer, ulcerative colitis and the like is made possible safely, rapidly and low-invasively, and the degree of precision is significantly improved.

With regard to such endoscopic observation, the tissue stain of the present invention may be directly disseminated in the gastrointestinal lumen or administered submucosally, or may be administered orally or intravenously. In this case, the luminal surface of the gastrointestinal tract and/or the interior of the cells in the gastrointestinal lumen are chromophilic.

### EXAMPLES

Next, the present invention will be described in more detail with reference to Examples, but the present invention is not intended to be limited by these Examples.

### Example 1

Measurement of an absorption spectrum and a fluorescence excitation spectrum of Fast Green FCF was performed. The Fast Green FCF (Wako Pure Chemical Industries, Ltd.) was adjusted to 1.0 mg/ml, and the light absorbance was continuously measured at a wavelength of from 200 to 750 nm using a spectrophotometer (manufactured by Shimadzu Corp., BioSpec-1600) to determine the wavelength at which the absorbance reached the maximum. The maximum absorption wavelength of 628 nm thus obtained was irradiated as an excitation wavelength, and the wavelength of the scattered light detected in the perpendicular direction to the light axis of the excitation light was measured using a fluorescence spectrophotometer (manufactured by Shimadzu Corp., RF-1500), thus to obtain a maximum excitation wavelength of 628 nm and a maximum fluorescence wavelength of 652 nm. FIG. 1 shows the maximum excitation wavelength and the maximum fluorescence wavelength measured with the fluorescence spectrophotometer.

As a result of performing the same test, Brilliant Blue FCF was found to have a maximum absorption wavelength of 629 nm, a maximum excitation wavelength of 619 nm, and a maximum fluorescence wavelength of 650 nm. Guinea Green B had a maximum absorption wavelength of 620 nm, a maximum excitation wavelength of 624 nm, and a maximum fluorescence wavelength of 635 nm. Patent Blue VF had a maximum absorption wavelength of 639 nm, a maximum excitation wavelength of 641 nm, and a maximum fluorescence wavelength of 654 nm. Light Green SF had a maximum absorption wavelength of 635 nm, a maximum excitation wavelength of 633 nm, and a maximum fluorescence wavelength of 652 nm. Alphazurine FG had a maximum absorption wavelength of 630 nm, a maximum excitation wavelength of 633 nm, and a maximum fluorescence wavelength of 643 nm. Therefore, the compound of Formula (1) was found to be excited by a light source in the red wavelength region and emit fluorescence.

### Comparative Example 1

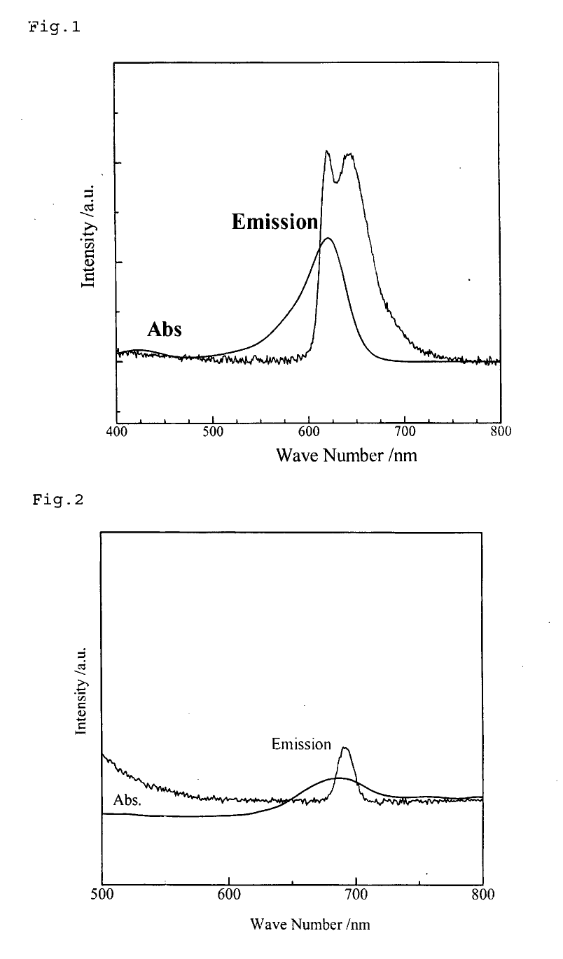

The fluorescence spectrum of IndoCyanine Green is shown in FIG. 2. It can be seen that the fluorescence intensity is lower than that of the Fast Green FCF.

### Example 2

In order to find the stain specificity of the stain, Fast Green FCF (Wako Pure Chemical Industries, Ltd.) was diluted to a 2.0 mg/mL preparation using a 0.1 M sodium phosphate buffer solution (pH 7), and observation was performed using a confocal microscope (Leica Microsystems Corp., TCS-SP2).

For the sample, a mouse was subjected to laparotomy to extract the large intestine, which was then cut into a piece of 1 cm at every edge and stained with the stain.

The conditions for observation with the confocal microscope were such that a He—Ne laser was irradiated at a wavelength of 633 nm, and the fluorescence was observed in the wavelength range of 650 to 750 nm. The diameter of the confocal pinhole was 1.00 airy, and an oil immersion lens of 63 magnifications was used.

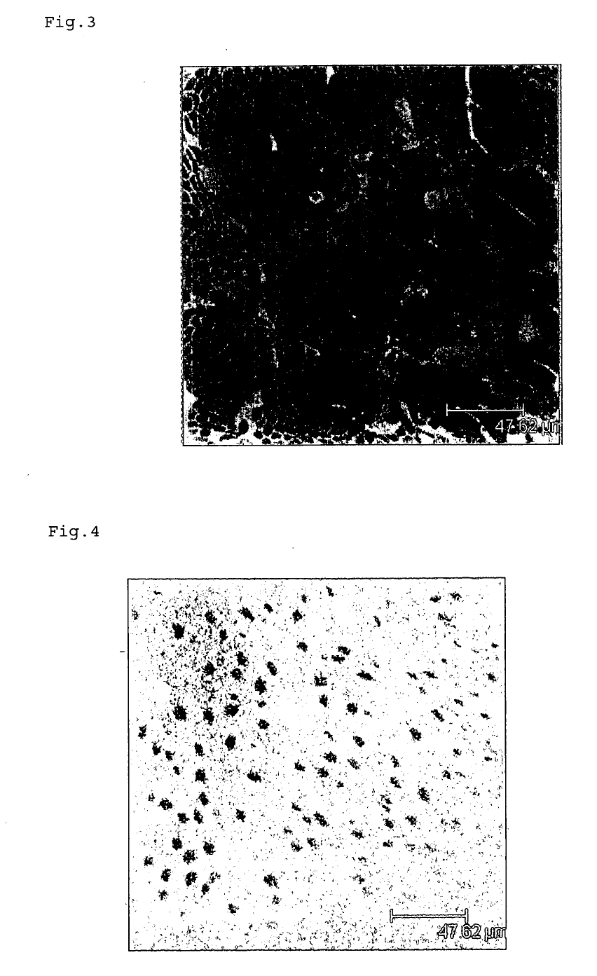

FIG. 3 shows the pictures taken with the confocal microscope. It was shown that the Fast Green FCF provided fluorescent contrast to the images of the intercellular space of the mucosal epithelium of the large intestine.

### Comparative Example 2

The stainability of the Fast Green FCF stained in Example 2 was compared with the stainability of Fluorescite (Alcon Corp.) which has been conventionally used.

Staining was performed by immersion in the same manner as in Example 2, excitation was induced at a wavelength of 488 nm, and the fluorescent images were observed.

The conditions for the confocal microscope were the same, except for the wavelength of the irradiated laser.

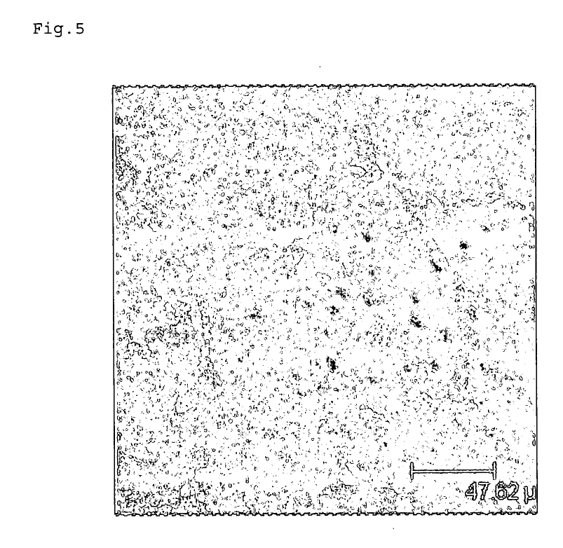

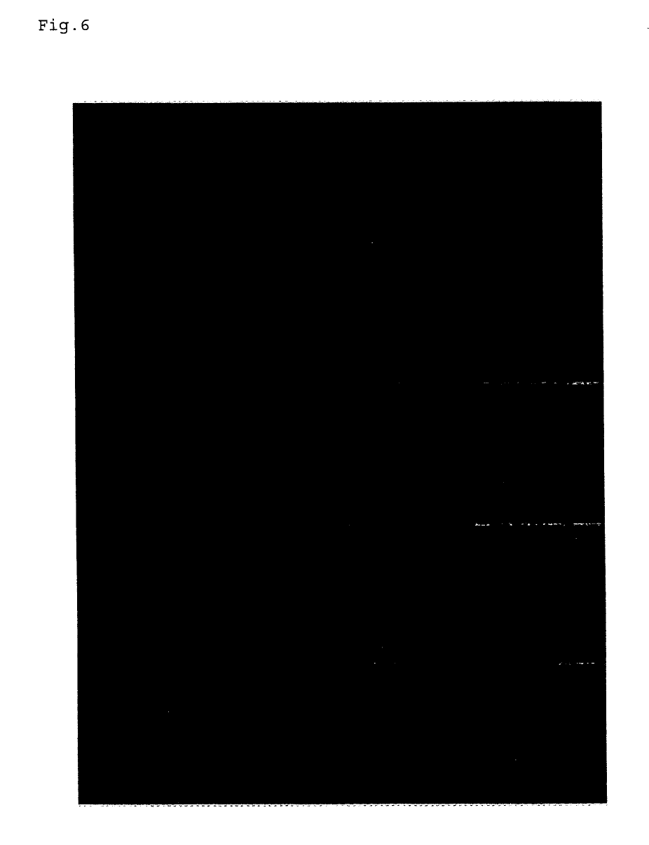

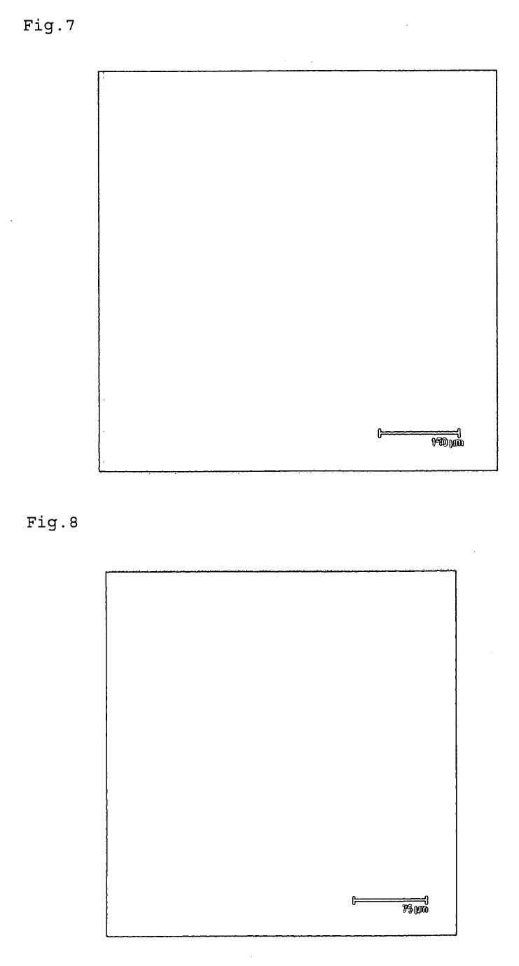

FIG. 4 shows a stained image obtained with Fluorescite, and FIG. 5 shows a double-stained image. From FIGS. 4 and 5, the feature of the Fast Green FCF as a fluorescent stain was found to be that the stain and Fluorescite had different chromophilic regions. It was found that the structure of cells constituting a tissue can be observed more clearly by using these two types of fluorescent stains simultaneously.

### Example 3

Because the observation capability of the images obtained with a confocal microscope changes due to stainability of Fast Green FCF (Wako Pure Chemical Industries, Ltd.), an evaluation of stainability was performed.

Fast Green FCF was prepared to 1.0 mg/mL using a 0.1 M sodium phosphate buffer solution (pH 6), and 100 μL of the stain solution was injected into the large intestine of a mouse (ddY, 11 weeks old, male). To stain the large intestine evenly while preventing outflow of the stain, the mouse was subjected to laparotomy, and the stain was injected while clipping the upward colon side and the downward colon side of the extracted large intestine with hemostatic clips.

The cross-section of the tissues in the lumen of the large intestine was observed with a confocal microscope (Leica Microsystems Corp., TCP-SP2).

The imaging conditions were such that the diameter of the confocal pinhole was 1.00 airy, and a lens of 20 magnifications was used.

FIG. 6 shows the images taken from the observation of the cross-sectional images at every 5.0 μm from the luminal surface. According to the present Example, Fast Green FCF provides good fluorescent contrast for the lamina propria mucosae which is beneath the epithelial cells of the large intestinal mucosa.

### Comparative Example 3

IndoCyanine Green (adjusted to 1 mg/mL with physiological saline), which is a medical contrast agent, was used to perform tissue staining. Mouse large intestine was stained by a perfusion method, and observation of an extracted specimen of the large intestine was performed using a confocal microscope (Leica Microsystems Corp., TCP-SP2). As a result of the observation of the inside of the tissue, the characteristics regarding stainability were similar to those of Fast Green FCF, but the fluorescence intensity was very low, and it was difficult to perform satisfactory observation.

### Example 4

The stainability and tissue penetrability of Fast Green FCF (Fast Green GCF, Wako Pure Chemical Industries, Ltd.) were checked by observation of a tissue specimen.

A tissue specimen of the large intestine extracted from a mouse, which had been stained in the same manner as in Example 3, was embedded in an OCT Compound (Sakura Finetek Corp., Tissue-Tek O.C.T. compound), and was frozen by rapidly cooling with dry ice. The frozen section was sliced into 6 μm to prepare frozen sections, and fluorescence observation was performed. The fluorescence observation was performed using a confocal microscope (Leica Microsystems Corp., TCP-SP2) (imaging conditions: using a lens of 40 magnifications, pinhole diameter 171.3 μm). The results are shown in FIG. 8.

### Comparative Example 4

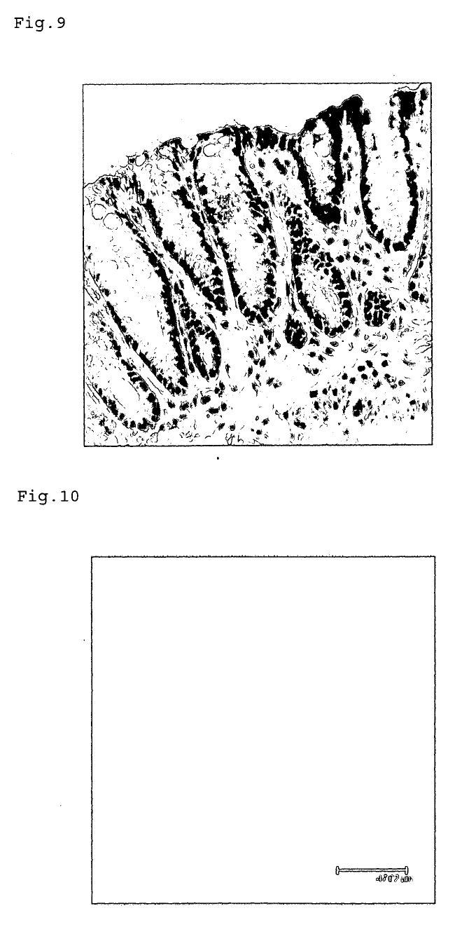

A tissue section which was similarly embedded in the OCT compound was HE-stained, and the observation result is shown in FIG. 9 as a Comparative Example. From this, it was found that Fast Green FCF provided good fluorescent contrast for the lamina propria mucosae which was beneath the epithelial cells in the large intestinal mucosa, and thus consistency was maintained.

### Example 5

An evaluation of the tissue stain composition of the present invention was performed for the stainability for different regions. Duodenum, large intestine and liver were extracted and observed.

Fast Green FCF was prepared to 10 mg/mL using physiological saline at pH 6.0. 100 μL of the prepared Fast Green FCF was administered to a mouse (ddY, 11 weeks old, male) through the tail vein, and the mouse was subjected to laparotomy after 5 minutes. The penetrability was observed by naked eyes, and the stainability and fluorescence were observed using a confocal microscope (Leica Microsystems Corp., TCP-SP2).

The duodenum was stained in deep-green color to an extent of being clearly visible by naked eyes.

Strong staining was achieved not in the cytoplasm but in the cellular membrane or intercellular space, so that the shape and size of the cells could be observed.

The conditions for the confocal microscopic observation were such that the diameter of the confocal pinhole was 1.00 airy, and an oil immersion lens of 63 magnifications was used. FIG. 10 shows a confocal microscopic image of an area 40 μm deep from the luminal surface.

The large intestine did not show color changes that were clear enough to be visible by naked eyes on the surface, but it was confirmed that the dye penetrated sufficiently to the extent that imaging by a confocal imaging system was possible.

The intercellular space near the luminal surface, and the lamina propria mucosae were well stained.

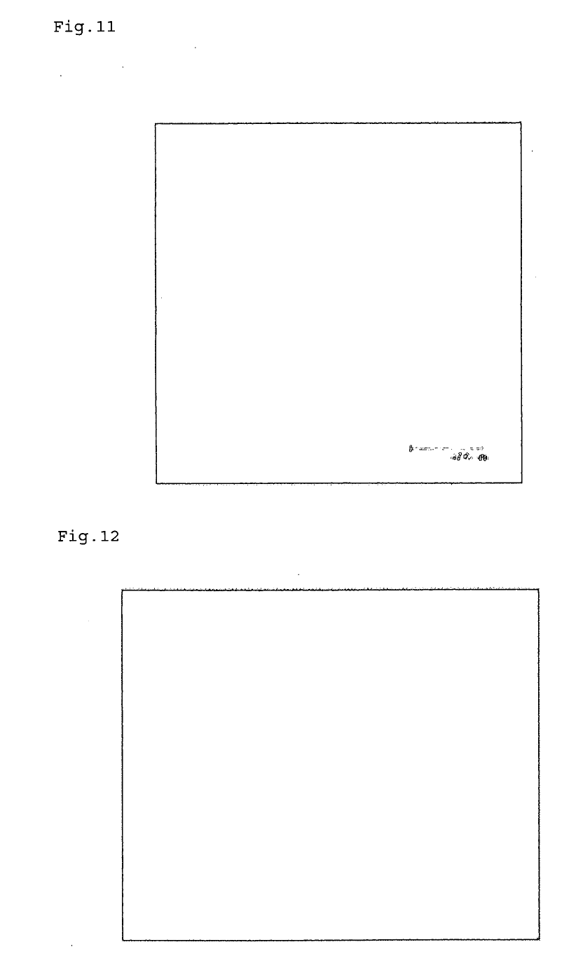

The conditions for the confocal microscopic observation were such that the diameter of the confocal pinhole was 1.00 airy, and a cross-sectional image of an area about 10 μm deep from the luminal surface, which image was taken using an oil immersion lens of 63 magnifications, is presented in FIG. 11.

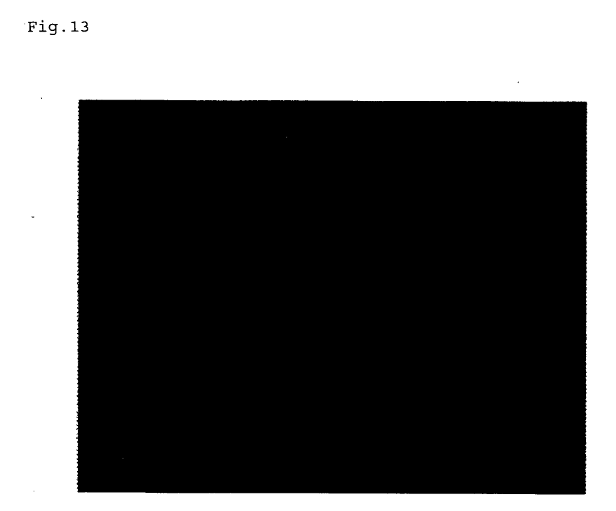

With respect to the liver, not only the intercellular space and cellular membrane as observed in the duodenum or large intestine but also the lobular parts were stained, so that the structure could be clearly observed. Furthermore, the surrounding septum was more strongly stained. The conditions for the confocal microscopic observation were such that the diameter of the confocal pinhole was 1.00 airy, and a cross-sectional image of an area 10 μm deep from the luminal surface, which image was taken using an oil immersion lens of 63 magnifications, is presented in FIG. 12.

According to the present Example, it was shown that Fast Green FCF conferred contrast such that observation of the small intestinal villi under a white light source became feasible. At the same time, it was also found that since Fast Green FCF provides clear fluorescent contrast for the shape of the epithelial cells under a confocal microscope, it is useful for the observation of tissue structures under any light source between a white light source and a red excitation light source. Also for the confocal microscopic observation of the liver, Fast Green FCF provided good contrast for the Glisson's capsule, which is an interlobular connective tissue in the liver, and this result is consistent with the observation result showing strong fluorescence of the connective tissue in the lamina propria mucosae in the large intestine. That is, according to the present Example, Fast Green FCF was shown to provide good fluorescent contrast for the connective tissue in the liver.

### Example 6

The esophagus part of a mouse (ddY, 11 weeks old, male) was extracted, stained using Brilliant Blue FCF, and observed with a confocal microscope (Leica Microsystems Corp., TCP-SP2).

The Brilliant Blue FCF used was prepared to 10 mg/mL using a 0.1 M sodium phosphate buffer solution (pH 7).

The imaging conditions were such that the pinhole diameter was 1.00 airy, and a lens of 20 magnifications was used.

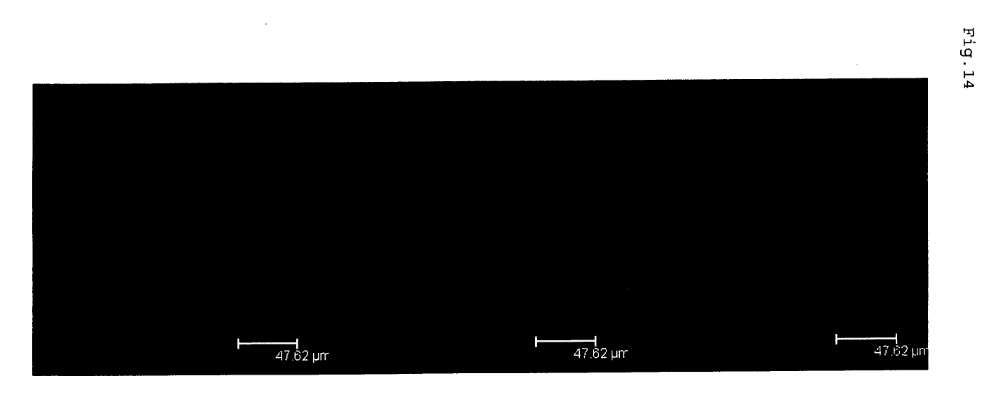

FIG. 13 shows cross-sectional images taken at every 5.0 μm from the luminal surface downward. Clear images up to 100 μm deep from the luminal surface could be obtained.

It was confirmed that keratinized squamous epithelial cells were lining up in the vicinity of the tissues surface layer, while squamous epithelial cells were lining up inside the tissues. According to the present Example, Brilliant Blue FCF was found to provide fluorescent contrast for the intercellular space of the esophagus epithelial cells.

### Example 7

A tumor tissue developed in the upper part of the ovary of a rat (F344/DuCrj, 15 weeks old, female) was extracted, and a cross-sectional image of the inside of the tissue was observed with a confocal microscope (Leica Microsystems Corp., TCP-SP2).

Fast Green FCF was prepared to 1.0 mg/mL using a 0.1 M sodium phosphate buffer solution (pH 7).

The tumor tissue of the rat was extracted and immersed in the prepared stain for 5 minutes to stain the tissue. The remaining unpenetrated portion of the Fast Green was washed off with a 0.1 M sodium phosphate buffer solution (pH 6), and observation was made using a confocal microscope.

The observation conditions were such that the pinhole diameter was 1.00 airy, and an oil immersion lens of 63 magnifications was used.

The images taken with the confocal microscope are shown in FIG. 14. FIG. 14 shows serial images taken at every 5.0 μm from an area 10 μm deep from the surface layer downward. According to the present Example, it was found that Fast Green FCF provided good fluorescent contrast for the connective tissue fibers in the tumor tissue.

### Example 8

To verify the stainability at a tumor part, Monascus Yellow was used together with Fast Green FCF to perform double staining. The Monascus Yellow was prepared to 1.0 mg/mL using a 0.1 M sodium phosphate buffer solution (pH 6).

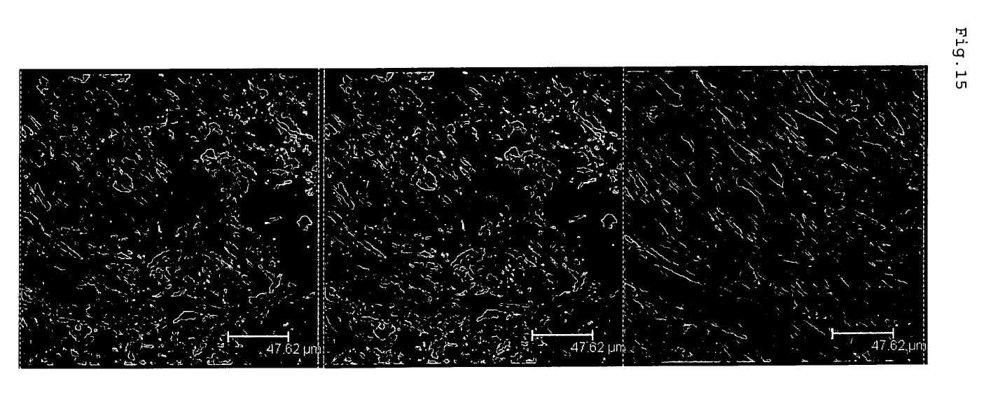

Confocal microscopic images are shown in FIG. 15.

Three images were taken in correspondence to FIG. 14 of Example 7, and show the same parts. It is clear from a comparison of the images that while Monascus Yellow stained the entirety of the cells (excluding the nuclei), Fast Green FCF slightly stained the cells but more strongly stained the fibrous part. According to the present Example, it was shown that Fast Green FCF had different staining properties than Monascus Yellow, so that clearer observation of the structure of the cells constituting a tissue can be made by using a combination of the Fast Green FCF and the Monascus Yellow.

### Example 9

Patent Blue was used for staining and observation with a confocal microscope. Patent Blue (Sigma-Aldrich Corp., Patent Blue VF) was prepared to 10 mg/mL with physiological saline, and was perfused through the heart of a mouse (ddY, 6 weeks old, male) to perform staining.

600 μL of the Patent Blue solution was used, and the limbs were bluish in color to the extent of being visible by naked eyes. The mouse was subjected to laparotomy to extract the large intestine, and an observation was made for a specimen with a confocal system.

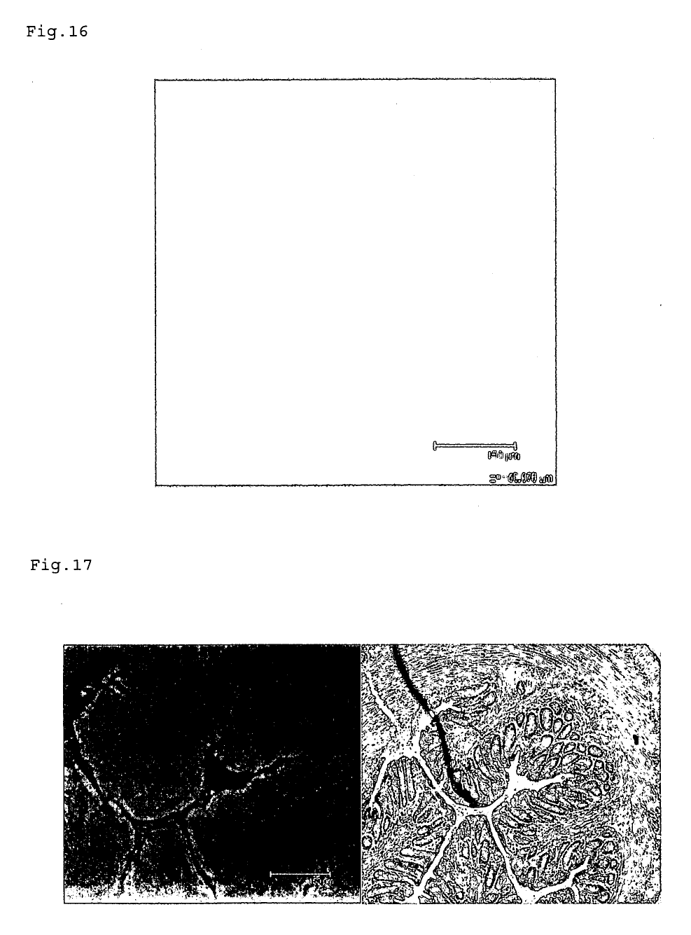

FIG. 16 shows a cross-sectional image of an area 60 μm deep from the luminal surface of the tissue. The imaging conditions were such that a lens of 20 magnifications was used, and the pinhole diameter was 1.00 airy. As the confocal microscope, TCP-SP2 manufactured by Leica Microsystems Corp. was used. From FIG. 16, it was determined that the lamina propria mucosae could be observed clear using Patent Blue.

### Example 10

A frozen section prepared by slicing the mouse large intestine stained in Example 9 to approximately 6 μm was observed under fluorescence to observe the stainability. An adjacent frozen section was used to perform HE staining and was compared to characterize the sites where fluorescent observation was possible by Patent Blue staining.

A confocal microscope was used for the observation, which was made at 20, 40 or 63 magnifications. FIG. 17 shows a picture taken using a lens of 20 magnifications and a pinhole diameter of 2.66 airy.

Since the section of the large intestine subjected to fluorescent observation showed strong fluorescence in the lumen, it can be said that the observation capability was high.

### Example 11

The gastrointestinal lumen was stained using Guinea Green, and the stainability was examined by observation of a frozen section.

Guinea Green (Sigma-Aldrich Corp., Guinea Green B) was prepared to 10 mg/mL with physiological saline, and was perfused through the heart of a mouse (ddY, 6 weeks old, male) in an amount of 600 μL.

Also, it was compared with another frozen section used to perform HE staining, and the sites capable of fluorescent observation by Guinea Green staining were characterized.

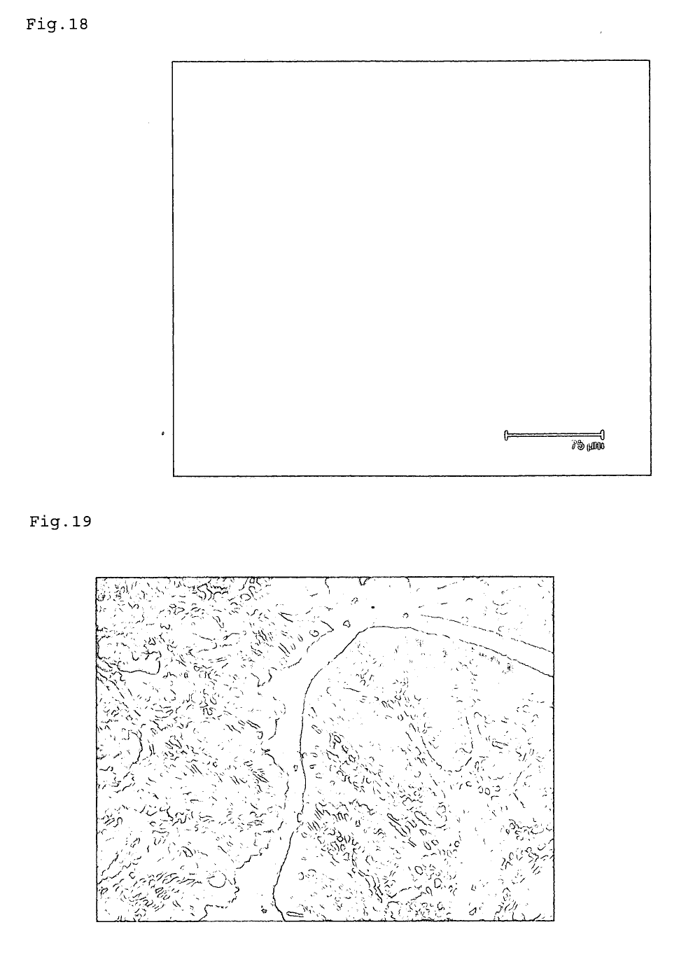

A confocal microscope was used for the observation, which was made at 20, 40 or 63 magnifications. FIG. 18 shows an image observed and taken under the conditions of a pinhole diameter of 1.00 airy using a lens of 40 magnifications.

FIG. 19 shows a HE-stained slice section of almost the same spot as that of the Guinea Green-stained section.

In this case, the lumen side exhibited strong fluorescence as in Example 10. As a result, the observation capability of the confocal system using Guinea Green was determined to be high.

What is claimed is:

1. A method of diagnosing with an endoscope, comprising administering a composition containing a compound represented by the following Formula (1): 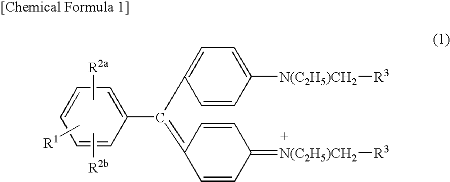
wherein R¹ represents a hydrogen atom or a hydroxyl group; R³ represents a phenyl group or methyl group, to which R^{2c} is bound; one to three of R^{2a}, R^{2b} and R^{2c} represent —SO₃M (wherein M represents an alkali metal atom or an alkaline earth metal atom), or —SO₃NH₄, and when one represents —SO₃⁻, the others represent hydrogen atoms;
and observing the tissue fluorescent stained by the composition using an endoscope.

2. The method according to claim 1, wherein the compound represented by the Formula (1) is selected from the group consisting of Fast Green FCF, Brilliant Blue FCF, Guinea Green B, Patent Blue VF, Patent Blue NA, Patent Blue CA, Light Green SF and Alphazurin FG.

3. The method according to claim 1, wherein the endoscope is a medical endoscope.

4. The method according to claim 1, wherein the endoscope is a fluorescent endoscope or a confocal endoscope.

5. The method according to claim 1, wherein the administration of the composition is achieved by oral administration, direct administration into the gastrointestinal tract, or submucosal administration.

6. The method according to claim 1, wherein the surface of the gastrointestinal lumen and/or the inside of the cells of the gastrointestinal lumen is stained.

7. A histofluorescent stain composition for endoscopy, comprising a compound represented by the following Formula (1) 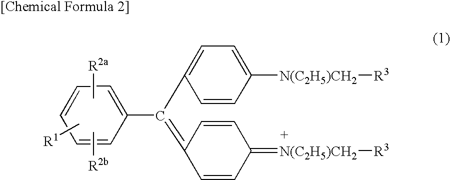
wherein R¹ represents a hydrogen atom or a hydroxyl group; R³ represents a phenyl group or methyl group, to which R^{2c} is bound; one to three of R^{2a}, R^{2b} and R^{2c} represent —SO₃M (wherein M represents an alkali metal atom or an alkaline earth metal atom), or —SO₃NH₄, and when one represents —SO₃⁻, the others represent hydrogen atoms.

8. The histofluorescent stain composition according to claim 7, wherein the compound represented by the Formula (1) is selected from Fast Green FCF, Brilliant Blue FCF, Guinea Green B, Patent Blue VF, Patent Blue NA, Patent Blue CA, Light Green SF and Alphazurine FG.

9. The stain composition according to claim 7, wherein the endoscope is a medical endoscope.

10. The stain composition according to claim 7, wherein the endoscope is a fluorescent endoscope or a confocal endoscope.

11. The stain composition according to claim 7, which is orally administered, directly administered into the gastrointestinal tract, or submucosally administered.

12. The stain composition according to claim 7, which stains the surface of the gastrointestinal lumen and/or the inside of cells of the gastrointestinal lumen.
